(19) United States
(12) Patent Application Publication
(10) Pub. No.: US 2009/0246035 A1
(43) Pub. Date: Oct. 1, 2009

(54) Pump Module Fluidically Isolated Displacement Device

(75) Inventor: Charles R. Patzer, Columbus, OH (US)

Correspondence Address: WOOD, HERRON & EVANS, LLP, 2700 CAREW TOWER, 441 VINE STREET, CINCINNATI, OH, 45202, US

(73) Assignee: SMITHS MEDICAL ASD, INC., Keene, NH (US)

(21) Appl. No.: 12/057,774

(22) Filed: Mar. 28, 2008

(51) Int. Cl. A61M 1/00; B23P 15/00; F04B 43/04; F04B 17/03; A61M 5/142; A61M 1/10

(52) U.S. Cl. 417/53; 29/888.02; 417/413.1; 604/153; 92/48

(57) ABSTRACT

A pump module for use in a medical fluid dispensing system is provided that includes a pump body having first and second portions where at least one of the first and second portions includes first and second fluid chambers. The module further includes at least one membrane operably associated with the fluid chambers, and first and second actuators operably associated with this membrane for the purpose of displacing the membrane and further for displacing fluid from the first and second chambers.

### FIELD

The present invention relates generally to pumps, and more particularly to pump modules for use in medical fluid dispensing systems.

### BACKGROUND

A variety of known pumps are used to dispense medical fluids. Syringes, which may act alone or in conjunction with a syringe pump, are widely used to dispense relatively small volumes of medical fluids, which can include high concentrations of medication. The maximum volume of syringes is typically about 60 mL. After this volume is dispensed, a caregiver must replace the depleted syringe to continue intravenous administration of a medical fluid. Accordingly, syringes do not lend themselves to applying large volumes of medication, dispensing of large volumes of blood, or the dispensing of high volumes of other fluid, such as saline, to burn patients for example.

When used in conjunction with a pump, the pump will automatically operate the single plunger or piston of the syringe. Typically, the plunger tip is made of a soft, compliant rubber. When the plunger is pushed to dispense fluid, the tip is compressed and forced to the outer wall of the syringe. “Stiction,” a term known in the art derived from the ability to stick in combination with static and dynamic friction, occurs when the piston is moved after being stationary. In such an intermittent operation, the force required to overcome the “stiction” and start the piston moving can cause a bolus, or positive pressure, of fluid to be dispensed and is undesirable.

Pumps that are used in systems to dispense large volumes of medical fluids include peristaltic pumps, diaphragm pumps, and single piston pumps. Although each type has been successfully used, they are subject to certain design and/or application challenges. For example, since the fluid flow passage in peristaltic pumps is normally open, fluid can inadvertently be supplied to the patient. This can occur if the tubing leading from a source of fluid, such as an IV bag, to the inlet portion of the pump is not clamped. Also, the continuous compression of the tubing defining the normally open flow path can result in tube fatigue, thereby necessitating replacement of the tube, which adds to the operational cost of the system.

Peristaltic pumps are affected by the hydraulic head height, resulting from the position of the source of fluid above the pump. This can, in turn, result in further inaccuracies with the flow rate of the pump.

Large volume single piston pumps are known, but do not exhibit fluid flow constancy. This is because for each pumping cycle a “dead time” occurs. That is, after a predetermined volume of fluid is pumped and the output valve is closed, the piston is retracted and the piston chamber must be refilled with fluid. This lack of flow constancy is undesirable because, for example, the half-life of certain medications can be on the order of seconds. If the medical fluid isn't delivered to and absorbed by the patient within one or two half-lifes, the effectiveness of the medical fluid is reduced for its intended use. Flow constancy is a particularly important consideration when high potency medical fluids are being dispensed.

Known diaphragm pumps used in large volume medical fluid dispensing systems include those having a single elastomeric diaphragm and an associated piston to deform the diaphragm and dispense the medical fluid. Diaphragm pumps of this type can also include elastomeric check valves that communicate with the pump inlet and outlet ports. The compliant nature of these check valves can lead to variations in the breaking pressure of the valves, i.e., the pressures required to open or close the valves, which in turn can result in flowrate accuracy issues. A lack in flow constancy due to fluctuations in flowrate of the medical fluid being delivered is undesirable for the same reasons discussed previously with respect to the lack of flow constancy caused by “dead time.” Another challenge associated with pumps having elastomeric diaphragms is that the diaphragm(s) deform during the fill cycle and store potential energy. This energy is released during the pumping cycle, which can again cause a bolus of fluid to be dispensed initially. This temporary spike in fluid flowrate also adversely affects flow constancy and is therefore undesirable.

Another known diaphragm pump used to dispense large volumes of medical fluids includes two elastomeric diaphragms that are pumped in alternating fashion. This pump does not include elastomeric check valves and the associated challenges. In some instances, as with a single piston diaphragm pump, the compliant, elastomeric diaphragms are pressurized during the fluid fill cycle causing them to deform and store energy. Accordingly, when the corresponding output valve is opened at the beginning of a pumping cycle, a bolus of fluid can be dispensed, even without the associated piston moving, which is undesirable. Thus, it would be desirable to establish a diaphragm pump that reduces the bolus effects.

Yet another challenge associated with medical fluid pumps is the requirement to replace the portion of the pump that is exposed to the fluid after a predetermined, relatively short period of time as a result of hospital procedures associated with infection control. This replacement must be accomplished in an expeditious and cost effective manner. The components of medical fluid dispensing systems that are exposed to, or wetted by, the fluid being dispensed include the fluid supply and discharge tubing and the portions of the pump that are exposed to the medical fluid. Due to the requirement of replacing these components after a relatively short period of time, there is a requirement for providing a pump module that can be replaced easily and in a cost effective manner.

### SUMMARY

In view of the foregoing and by virtue of the present invention, a pump module for use in a medical fluid dispensing system comprises a pump body having first and second portions. First and second fluid chambers are formed in either, or both, of the first and second portions. Either, or both, of the first and second portions further includes a fluid flow network for supplying a fluid from a fluid source to the fluid chambers and then dispensing the fluid from the fluid chambers during operation of the pump module. There is at least one membrane operably associated with the first and second fluid chambers, and first and second actuators operably associated with this membrane and with the first and second chambers, respectively. Displacement of the membrane by the actuators will result in displacement of fluid from the fluid chambers.

The first portion of the pump module can be a back portion and the second portion can be a cover portion. The fluid chambers can be formed in the back portion. The membrane can be disposed between the back and cover portions. The cover portion can have first and second openings, which correspond to the first and second fluid chambers. The first and second openings permit the first and second actuators to displace the membrane. The first and second actuators have first and second fluidically isolated displacement devices, respectively, which contact the membrane to displace fluid from the first and second fluid chambers.

The displacement devices can be plungers.

The first and second fluid chambers can be recesses within the back portion.

The pump body can be constructed from a non-compliant material.

The first and second actuators can be independently operable from one another.

The back portion of the pump can include at least one push point. The push point can interrupt the fluid flow in the fluid flow network. The cover portion can include an opening corresponding to the push point. A third displacement device can be operably associated with the membrane at the push point and the opening.

The pump body can include at least one fluid valve, which can interrupt the fluid flow in the fluid network.

The first and second fluid chambers can be fluidically sealed by the membrane. The membrane can include first and second membranes where the first and second membranes can be associated with the first and second chambers, respectively. The first and second fluid chambers can be fluidically sealed with the membrane and an O-ring.

The first and second fluid chambers can include an inner diameter constructed such that there is complete contact with the outer diameter of the displacement device.

According to a second aspect of the present invention, a method of manufacturing a pump module for use in a medical fluid dispensing system is provided comprising using a non-compliant material to form the pump body having first and second portions; forming first and second fluid chambers in at least one of the first and second portions; forming a fluid flow network in at least the first and second portions for supplying fluid from a fluid source to the fluid chambers and dispensing the fluid from the fluid chambers during operation of the pump; and forming first and second openings in the other of the first and second portions where the openings correspond to the first and second chambers, respectively; positioning a membrane between the first and second portions to fluidically seal the fluid chambers and the fluid flow network; providing first and second actuators positionally associated with the first and second fluid chambers, respectively, and operably associated with the membrane for displacing the membrane and displacing fluid from the first and second chambers; and positioning the membrane between the first and second portions and securing the first and second portions together.

The method can further comprise forming at least one push point within the fluid flow network. Further, the method can include a step of forming an inner diameter of the first and second openings and an outer diameter of the first and second actuator such that there is no gap between the inner and outer diameters.

Additionally, the method can include a step of providing tension to the membrane until the back and cover portions are adjoined such that the tension is maintained.

According to a third aspect of the present invention, a method for pumping fluid in a medical fluid dispensing system is provided comprising providing a pump having first and second portions where at least one of the first and second portions include first and second fluid chambers and a fluid flow network and where at least one membrane is disposed between the first and second portions. A fluid is supplied through the fluid flow network to the fluid chambers. The membrane is displaced at the fluid chambers and thereby displaces the fluid from the fluid chambers into the fluid flow network and out of the pump.

The method can further comprise initiating a first pumping cycle to displace at least a portion of the fluid out of the first fluid chamber, and, before the first pumping cycle is complete, initiating a second pumping cycle to displace at least a portion of the fluid out of the second fluid chamber. The stop of pumping can further comprise the refilling of the first fluid chamber after the first pump cycle and during the second pump cycle. The method can further include filling the second fluid chamber after completion of the second pump cycle and during a third pump cycle.

### DRAWINGS

These and other features, aspects, and advantages of the present invention will become better understood with regard to the following description, appended claims, and accompanying drawings wherein:

FIG. 1 is a schematic illustration of a system for dispensing medical fluids intravenously to a patient, which incorporates a pump according to the principles of the present invention;

FIG. 2 is a perspective view of the pump shown schematically in FIG. 1;

FIG. 3A is a cross-sectional view of the pump module; FIG. 3B is a perspective view of the same pump module as in FIG. 3A; FIG. 3C is a cross-sectional view of the first or second portions of the pump module shown schematically in FIG. 2 and according to one embodiment of the present invention with the associated displacement device;

FIG. 4 is a side elevation view of the pump shown in FIGS. 2 and 3;

FIGS. 5A through 5D are a series of front elevation views of a portion of the pump shown in general cross-section in FIG. 3 illustrating the positions of the pump push points during various phases of operation of the pump;

FIGS. 6A through 6D are a series of front elevation views of a portion of the pump shown in general cross-section in FIG. 3 illustrating the positions of the pump valves during various phases of operation of the pump;

FIG. 7 is a schematic representation of a control system that can be incorporated in the pump shown in FIGS. 2, 3, 4A-4B, 5A-5D; and 6A-6D; and

FIG. 8 is a cross-sectional view of the pump module having a stop-cock actuator assembly.

### DESCRIPTION

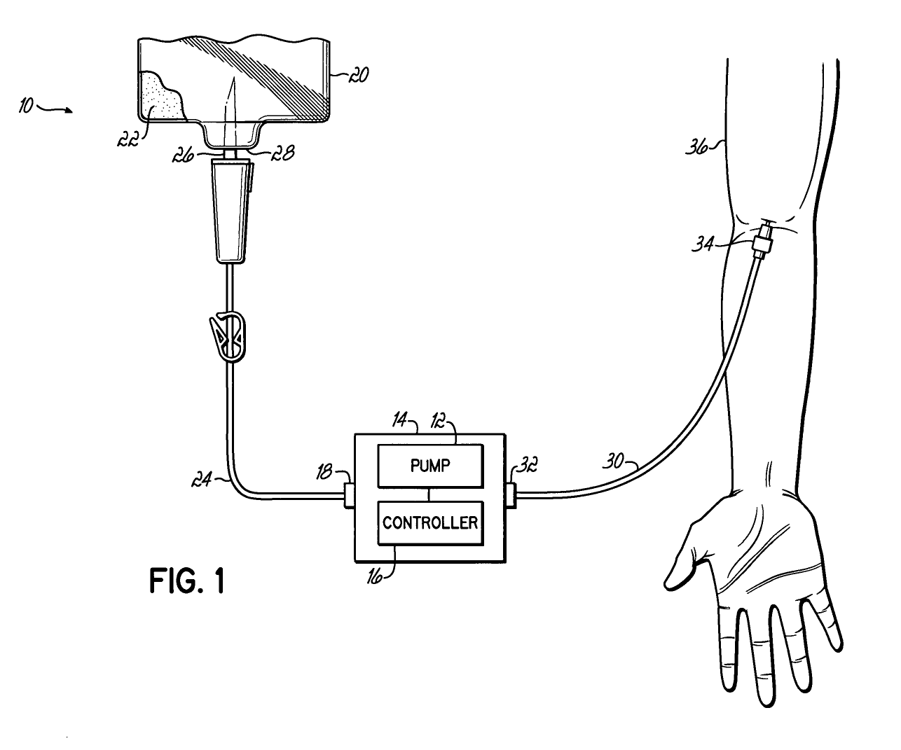

Referring now to the drawings, FIG. 1 illustrates a system 10 for dispensing medical fluids intravenously to a patient, where the system 10 is incorporating a pump 12 in accordance with the principles of the present invention. Pump 12 can be disposed within an enclosure 14 and can be electrically coupled to a controller 16, which can also be disposed within the same enclosure 14, and that can control the operation of pump 12.

A fluid inlet (not shown in FIG. 1) of pump 12 is fluidically coupled to a source of fluid to be dispensed to the patient. A suitable source of fluid may comprise a bag 20, commonly referred to as an IV bag, containing a fluid 22 therein. The fluid 22 can comprise a variety of medications and/or other fluids, such as saline solution, as is known in the art. The system 10 further includes a first section of tubing 24 that can comprise a single piece of tubing or multiple pieces of interconnected tubing. Tubing 24 can pass through a tubing inlet 18 of enclosure 14 and be fluidically coupled to a fluid inlet (not shown in FIG. 1) of pump 12 by one or more conduits and fluid connectors (not shown). The opposite end of tubing 24 can terminate in a spike 26 adapted to pierce a port 28 of the bag 20.

System 10 also includes a second section of tubing 30 that can comprise a single piece of tubing or multiple pieces of interconnected tubing. Tubing 30 can pass through a tubing outlet 32 of enclosure 14 and be in fluidic communication with a fluid outlet (not shown in FIG. 1) of pump 12 by one or more conduits and fluid connectors (not shown). The opposite end of tubing 30 can terminate in a catheter 34 inserted intravenously into an arm 36 of a patient.

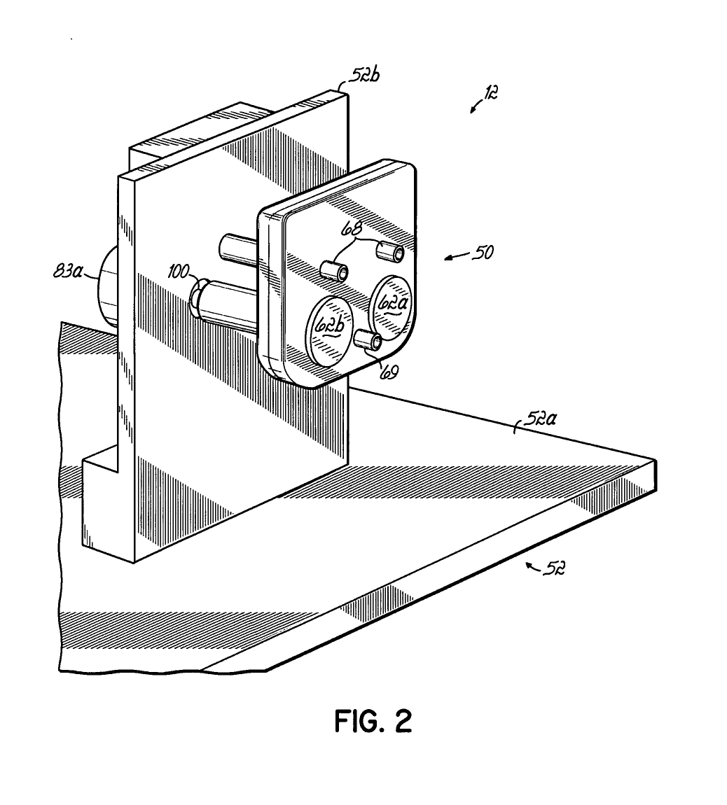

Referring now to FIGS. 2-4, the pump 12 shown schematically in FIG. 1 is further illustrated. Beginning with FIG. 2, pump 12 is a displacement pump and includes a pump body 50 that is adapted to be mounted to a stationary structure, such as support structure 52. In the illustrated embodiment, support structure 52 further includes a base plate 52a and a vertically extending member 52b, extending upwardly from the base plate 52a. However, the pump body 50 can be mounted to a wide variety of stationary structures having other configurations. As illustrated, the pump 50 is secured to the vertically extending member 52b by the actuators (the primary purpose of these actuators is described in detail below). Alternatively, the pump body 50 may be secured to the vertically extending member 52b by a plurality of conventional fasteners, such as bolts that extend through sleeves and into or through the vertically extending member 52b. However, pump body 50 can be mounted to the structure 52 in any other suitable manner.

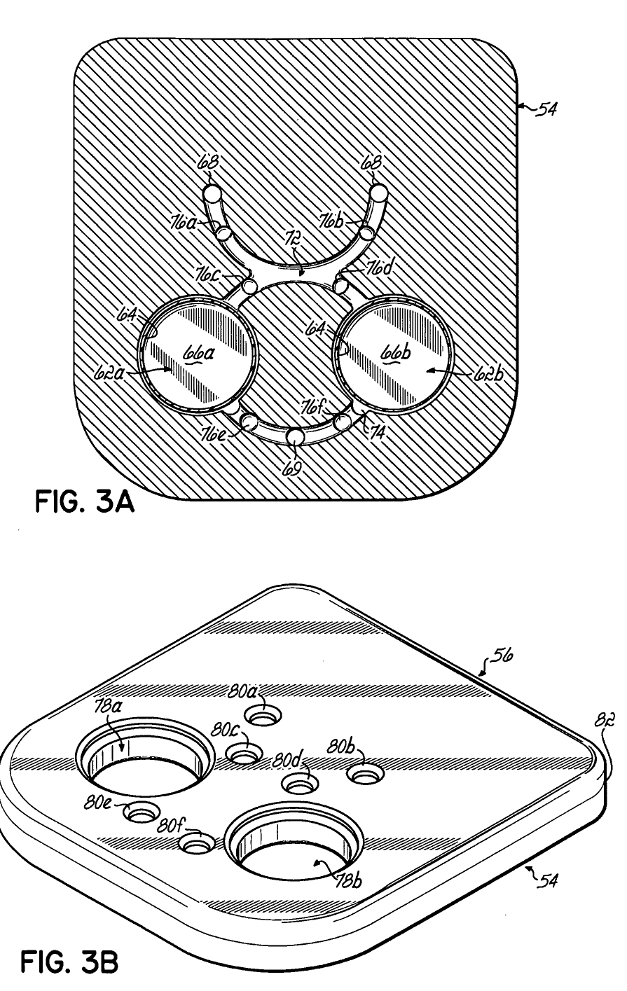

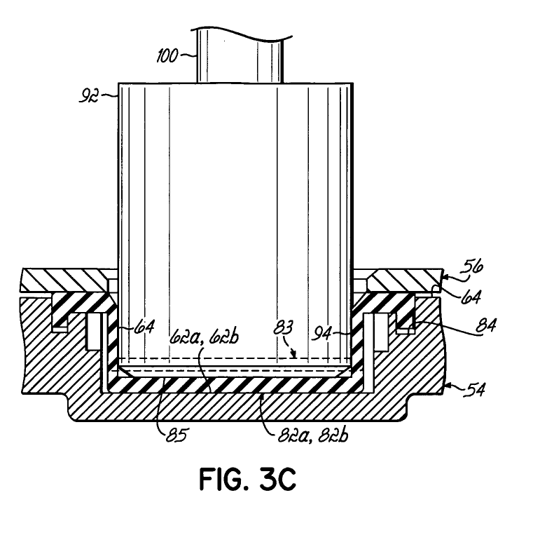

Pump body 50 includes first and second portions and can be made of a non-compliant material. Examples of suitable materials include various plastics such as an acrylic material or polycarbonates. The first and second portions can be, for example and as illustrated in FIG. 3, a back portion 54 and a cover portion 56 where the back portion includes first and second fluid chambers 62a, 62b. Fluid chambers 62a and 62b can be formed as recesses within either or both of the first and second portions by injection molding or other suitable manufacturing processes. While FIG. 3 illustrates the back portion 54 including the fluid chambers 62a, 62b, it would be readily appreciated the interchangeable nature of the first and second portions of the pump body 50. Each of the fluid chambers 62a, 62b includes an interior surface 64 defining first and second interior cavities 66a, 66b, respectively, formed in pump body 50 such that each of the chambers 62a, 62b is suitable for receiving a fluid as subsequently discussed. At least one fluid inlet 68 (illustrated herein with two fluid inlets) can be formed in pump body 50, with each disposed near one end of one of the chambers 62a, 62b. At least one fluid outlet 69 can be formed in pump body 50, disposed at an opposite end of the chambers 62a, 62b with fluid discharging therefrom as subsequently discussed.

The first and second fluid chambers 62a, 62b molded within the back portion 54 define first and second interior cavities 66a, 66b, respectively. While the interior cavities 66a, 66b are illustrated herein as having a generally circular shape defined by the interior surface 64, the interior cavities 66a, 66b may take on a shape that is best suited for a particular embodiment to be implemented and would be readily adaptable by one skilled in the art of manufacturing displacement pumps. Each of the fluid chambers 62a, 62b further includes at least one offset 74 so as to permit fluid communication between the fluid chambers 62a, 62b and the fluid flow network 72. That is, the interior surface 64 of fluid chambers 62a, 62b include an offset 74 extending transverse and outwardly beyond the interior cavity 66 and toward the fluid flow network 72. In this way, fluid may enter into or be displaced from the fluid chambers 62a, 62b in a manner to be discussed below. While offsets 74 are illustrated as having a general half-circular shape, it would be understood that such a shape is not limiting. Further, and also as illustrated, there may be as many offsets 74 as is necessary to provide fluidic communication between the chambers 62a, 62b and the fluid flow network 72.

While fluid chambers 62a and 62b are identically shaped and generally cylindrically shaped in the presently illustrated embodiment, it is conceivable and within the scope of the present invention that fluid chambers 62a and 62b can have shapes other than that shown or that they can have shapes that are not identical to one another.

Pump 12 further includes a pair of fluid displacement devices 92 (shown in FIG. 3C). The fluid displacement devices 92 are mechanically coupled to pump body 50 and are operably extendable into one of the fluid chambers 62a and 62b, wherein fluid is displaced out of the corresponding one of chambers 62a, 62b. Additionally, the fluid displacement devices are disposed in sealing engagement with the pump body 50, as subsequently discussed.

The fluid flow network 72 may further include at least one push point 76, but as illustrated includes six separate push points 76a-f. These push points, as in FIG. 3, comprise a slightly recessed portion within the fluid flow network 72 in the back portion 54 of the pump body 50 such that when a press point is pressed, the flow of fluid within the fluid flow network 72 is interrupted. Additional detail with respect to the interruption of fluid flow is below.

Continuing now to FIG. 3B, the cover portion 56, i.e. the second portion, of the pump body 50 is shown. The cover portion 56 may also be made of a non-compliant material, as described previously, and of the same general shape as the back portion 54. As illustrated in FIG. 3B, the cover portion 56 includes first and second openings 78a, 78b which spatially correspond with the first and second fluid chambers 62a, 62b. That is, once the back portion 54 and cover portion 56 are aligned, as in FIG. 3B, the first and second openings 78a, 78b of the cover portion 56 will be placed directly above the first and second fluid chambers 62a, 62b of the back portion 54. The first and second openings 78a, 78b can be formed by injection molding during manufacture of the cover portion 56, created subsequent to the molding process, or in another manner suitable and known in the art.

The cover portion 56 may further include at least one orifice 80 corresponding to the at least one push point 76 of the back portion 54. The operation of the push points 76 and associated orifice 80 is fully explained below.

A membrane 82 disposed between the back portion 65 and cover portion 56, fluidically seals the fluid chambers 62a, 62b and the fluid flow network 72 of the back portion 54. While one membrane may provide for all of the aforementioned fluidic seals, as shown in FIGS. 3A and 3B, in another embodiment, for example in FIG. 3C, it is possible to include first and second membranes 82a, 82b associated with first and second fluid chambers 62a, 62b, respectively.

In FIG. 3C, the membranes 82a, 82b associated with each fluid chamber 62a, 62b, respectively, and are held tightly in place once the cover portion 56 and the back portion 54 are secured together. In the illustrated embodiment, the back portion 54 includes a rim 84 about the perimeter of the interior surface 64 of the fluid chamber 62a, 62b at the surface adjoining the cover portion 56. This rim 84 may receive a portion of the membrane 82 with or without an O-ring (not shown). The O-ring is positioned between the membrane and the cover portion 56 such that the membrane 82 is held tightly and fluidically sealing the fluid chambers 62a, 62b. It should be appreciated that other manners of sealing the membrane 82 to the fluid chambers 62a, 62b would be known in the art. Additionally, it would be known as to how to include an O-ring seal in combination with an embodiment having a single membrane 82, as illustrated previously.

As illustrated, the displacement device 92 engages the membrane 82 to cause the deflection of the membrane 82 into the fluid chamber 62. Once the displacement device 92 is withdrawn, the compliant nature of the membrane 82 would cause the membrane to return to a non-deflected position 83.

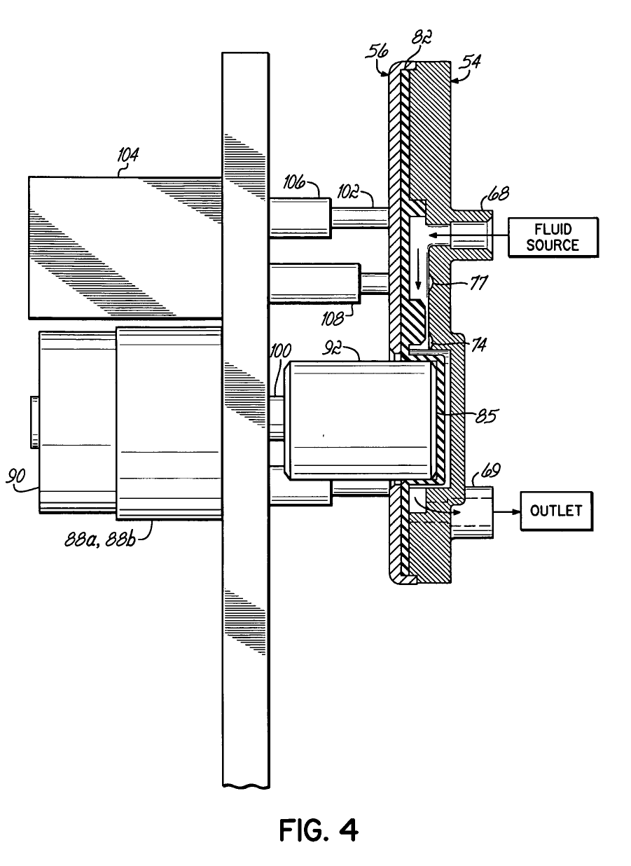

Turning now to FIG. 4, pump 12 further includes first and second actuators 88a and 88b. The first and second actuators 88a, 88b are mechanically coupled to the pump body 50 and are operably associated with membrane 82 as to displace the membrane 82 and thereby displace fluid from first and second fluid chambers 62a, 62b. First and second actuators 88a, 88b are fluidically isolated from the pump body 50, as subsequently discussed.

In one embodiment, the actuators 88a, 88b may include a stepper motor 90 and a displacement device 92 in operable engagement to the membrane 82. The displacement device 92 is extendable by actions of the stepper motor 90 so as to abut and displace membrane 82 for the purpose of displacing fluid from fluid chambers 62a, 62b without penetrating the fluidically sealed pump body 50. While the displacement device 92 illustrated in FIG. 3C is a plunger, other devices or shapes and sizes other than the illustrated plungers. It will be appreciated that when the displacement device 92 engages the membrane 82 that there should be little to no air gap at this interface 85 to allow for compliance of the materials. Otherwise, gaps or unsupported materials within the interface 85 will negatively affect the accuracy of the pump 12. As is illustrated, the first and second displacement devices 92a, 92b correspond to the first and second openings 78a, 78b, respectively.

The displacement devices 92, along with the associated actuators 88a, 88b are positioned upon the vertically extending member 52b so as to securely correspond to the location of the first and second openings 78a, 78b as well as the first and second fluid chambers 62a, 62b, as shown in FIG. 4. As illustrated, each stepper motor 90a, 90b is secured to the vertically extending member 52b of structure 52. The motors 90a, 90b can be secured to the vertically extending member 52b by any conventional means.

Turning again to FIG. 3C, wherein the fit between the displacement device 92 and the fluid chambers 62a, 62b is shown. Here, the displacement device 92 is well fitted to the interior surface 64 of the fluid chambers 62a, 62b. Specially, the inner diameter 94 of the fluid chambers 92a, 92b and the outer diameter 96 of the displacement device 92 are constructed to be tight fitting such that no air gaps exist at the interface 85 and to ensure complete and full transfer of displacement and the most efficient fluid displacement.

Each of the actuating devices 88a, 88b further includes a coupling 100 that is secured to a corresponding displacement device 92. This could be accomplished by passing a setscrew through a hole formed in coupling 100, until the setscrew is disposed in contacting engagement with the displacement device 92. Accordingly, as the coupling 100 is translated in or out, during operation of the stepper motor 90, the displacement device 92 moves, responsively, in or out with the coupling 100.

Because the displacement device 92 remains fluidically isolated from the pump body 50, there is no further requirement for extensive sealing members engaged between the displacement device 92 and the fluid chambers 62a, 62b. As such, it is completely possible to disengage the fluid inlets 68 and fluid outlets 69 such that the back portion 54, cover portion 56, and membrane 82 may be considered a disposable pump body 50.

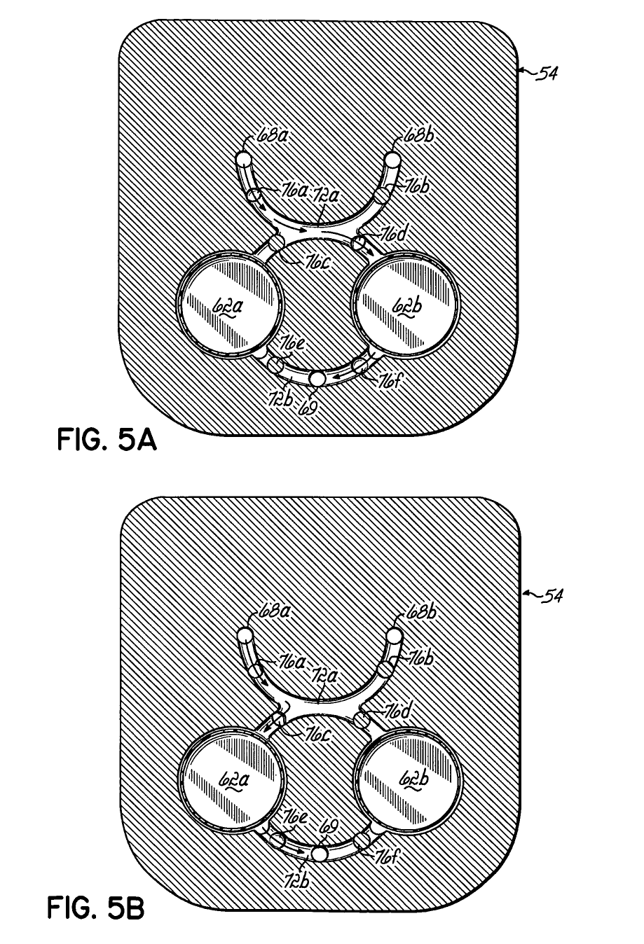

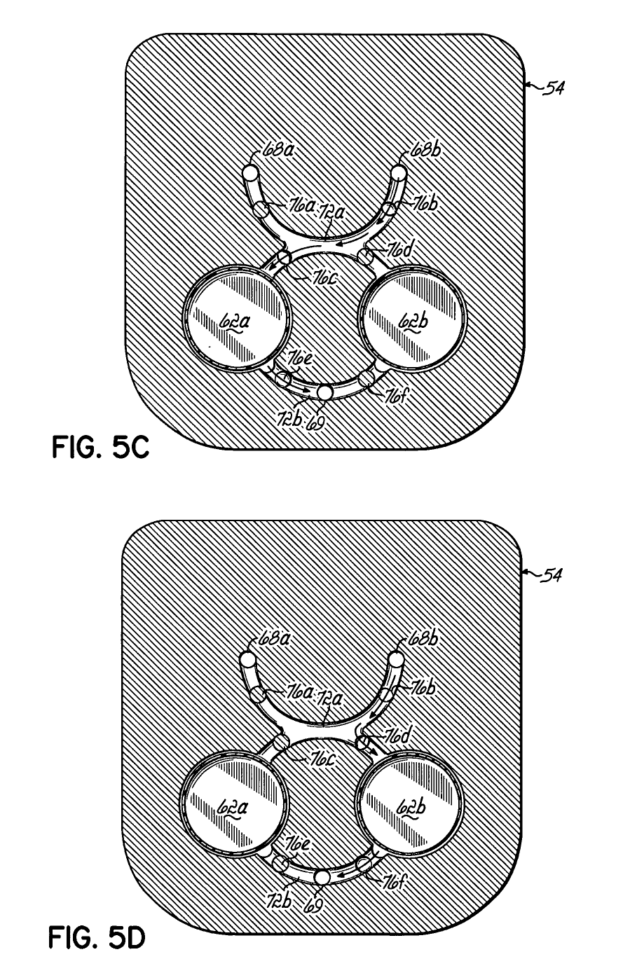

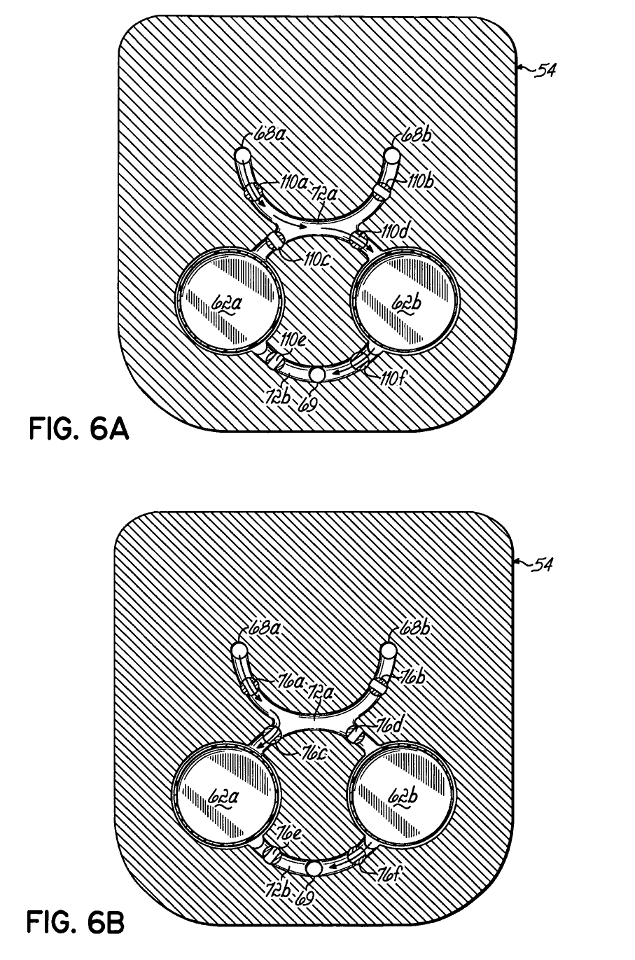

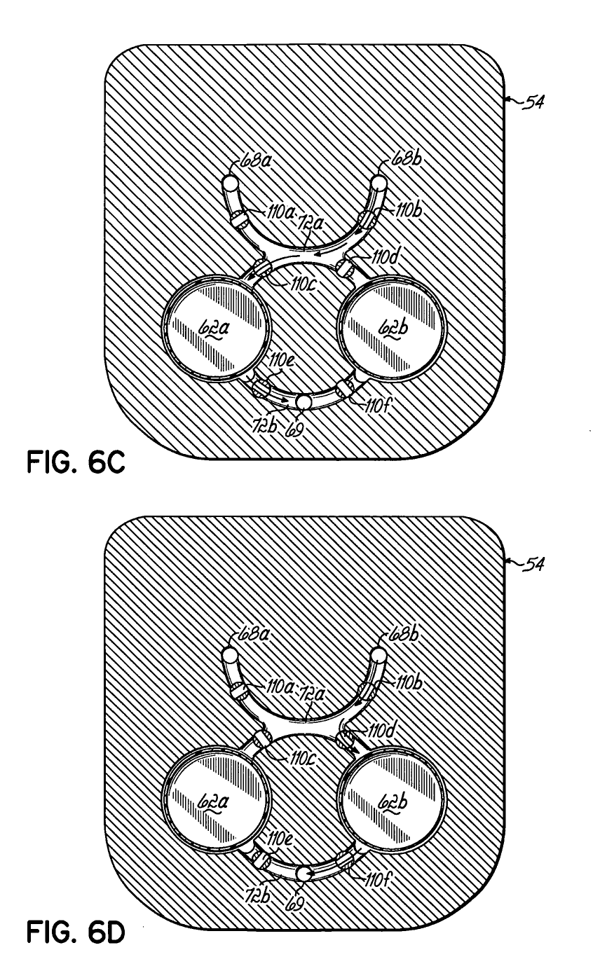

Turning now to FIGS. 5A to 5D, pump 12 further includes a fluid flow network, indicated generally at 72 in FIGS. 5A-5D, that is formed in the pump body 50 and is operable for supplying fluid from a source of fluid, such as the IV bag 20 (see FIG. 1), to the fluid chambers 62a, 62b, and for dispensing the fluid from the chambers 62a, 62b out of the pump body 50 during operation of pump 12. Fluid flow network 72 can be formed in at least one of the first and second portions of the pump body 50 by injection molding. In this illustrated embodiment, the pump body 50 includes two inlets 68 and four push points 76a-76d disposed within an influx fluid flow network 72a extending between the inlets 68 to first and second fluid chambers 62a, 62b. An efflux fluid flow network 72b then extends from the first and second chambers 62a, 62b to the one outlet 69, wherein two push points 76e, 76f are formed within the efflux fluid network 72b. While push points are specifically illustrated here, FIGS. 6A-6D will illustrate the replacement of the push points 76 with non-displacement style valves. The use of non-displacement valves would further aid in maintaining a desired flow constancy of pump 12 and is illustrated in FIGS. 6A-6D.

In the present embodiment, as illustrated in FIGS. 5A through 5D, the flow of fluid traversing the fluid flow network 72 can be selectively altered by push points 76a through 76f. Particularly, each push point 76 includes a recessed portion 77 within the fluid flow network 72 of the back portion 54 and can be accessed via a corresponding opening 80 (not shown) of the cover portion 56. The fluid flow network 72 is then fluidically sealed within the pump body 50 by membrane 82. As was illustrated and described in the operation of the stepper motor 90 and displacement device 92, a similar device may be included in the pump body 50 with respect to the push points 76. That is, pressure devices 102 are mechanically coupled to an actuator 104 and operably associated with the membrane 82 at each push point 76. In operation, the pressure devices 102 may operate in a manner similar to the displacement device 92 associated with the fluid chambers 62a, 62b, that is, with separate actuators 104 for each pressure device 102. An appropriate pressure device 102 may include a dowel rod, a plunger, a piston, or other similar device capable of reversibly interrupting the fluid flow within the fluid flow network 72. During operation of pump 12, the actuators 104 corresponding to each pressure device 102 move from a retracted position 106 to an extended position 108, wherein during the extended position 108 the membrane 82 is deflected such that membrane 82 contacts the recessed portion 77 of the push point 76 and the fluid flow is interrupted, i.e. closed off. As illustrated, each of the actuators 104 may include a stepper motor, rotational actuator, or other mechanism such as to provide the retracted 106 and extended 108 positions.

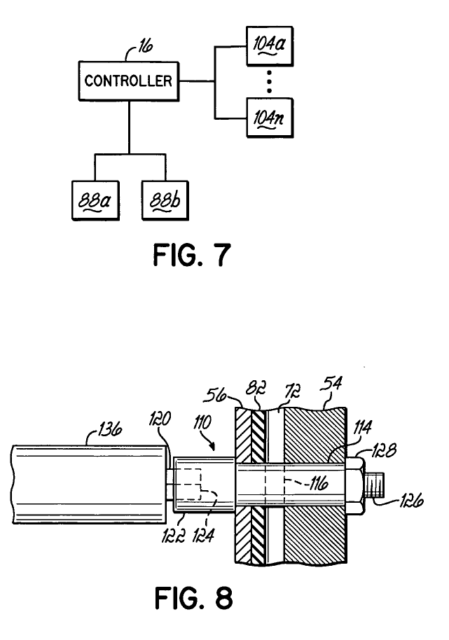

As shown in FIGS. 7A and 7B, a controller 16 controls the operation of the actuators 88a, 88b along with the associated displacement devices 92 in addition to the separate actuators 104 of pressure devices 102. The controller 16 can be programmed to operate the actuators 88a, 88b and pressure devices 102 to achieve a desired flow pattern throughout the pump. Actuators 88a, 88b are operated independent of one another while the actuators 104 of the pressure devices 102 operate independent of one another. It would also follow that actuators 88a, 88b operate independent of actuators 104 of the pressure devices 102. This permits fluid to be pumped out of either one of the fluid chambers 62a, 62b separately, but also permits the fluid to be pumped out of the fluid chambers 62a, 62b simultaneously, as is required to maintain a constant flow of fluid discharge through the outlet 69 of the pump body 50.

Referring again to FIGS. 5A-5D, during an initial phase, or cycle, in operating the pump 12, the second of the fluid chambers 62b is filled with the fluid to be dispensed, while fluid is displaced out of the other, first fluid chamber 62a, and through the outlet 69 into a tubing section, such as tubing section 30 of FIG. 1. That is, fluid enters the pump body 50 via fluid inlet 68a. The pressure device 102 associated with push points 76b and 76c, though not shown in FIG. 5A, are activated to the extended positions, thus interrupting flow of fluid through each respective point. Accordingly, fluid is supplied via a first fluid inlet 68a, will traverse the influx fluid flow network 72a such that this first fluid fills the second fluid chamber 62b. In another situation, fluid enters the pump body via the second inlet 68b while the pressure devices 102 (not shown in FIG. 5B) associated with push points 76a and 76c, are activated to the extended positions. Accordingly, it is possible to fill the second fluid chamber 62b with a fluid supplied by either the first or second inlets 68. Further, it would be appreciated that the embodiment according to FIGS. 5A and 5B permit the usage of two different fluid sources, wherein a first fluid source is fluidically connected to a first inlet and a second fluid source is fluidically connected to a second inlet. However, it would likewise be possible to include the same fluid source to both the first and second fluid inlets or have only one fluid inlet.

Activation of the first displacement device 92a causes displacement of membrane 82 and thus the fluid within the first fluid chamber 62a will be displaced from the first fluid chamber 62a. Because pressure device 102e is not activated into the extended position, fluid flow is not inhibited and thus may freely move from the first fluid chamber 62a to the output 69 via the efflux fluid flow network 72b. Activation of the pressure device 102f associated with push point 76f prevents unintentional back-fill into the second fluid chamber 62b or alternatively prevents the leaking of fluid when chamber 62b is filling.

When the displacement device 92a associated with the first fluid chamber 62a has reach the end of its stroke, or translation, push point 76e is activated by extending the pressure device 102e and causing the interruption of the fluid flow from the first fluid chamber 62a. Depending on the fluid to be used in filling the first fluid chamber 62a, pressure devices 102a, 102b, and 102c are in the retracted positions. Displacement device 92a is also retracted so that fluid chamber 62a is refilled with fluid by a volume equal to the volume of the portion of displacement of the membrane 82 by the displacement device 92a.

According to FIG. 9, the controller may activate the associated pressure devices and actuators such that the pump body 50 is in a state as illustrated either FIG. 5C or 5D. FIGS. 5C and 5D illustrate the filling of the first fluid chamber 62a in a manner similarly illustrated in FIGS. 5A and 5B with same or different fluid sources. Further, as briefly alluded to above, the filling of a first fluid chamber 62a may be followed by, or simultaneous to, the displacement of fluid from the second fluid chamber 62b. Simultaneous pumping out of both of the fluid chambers 62a, 62b may continue for a relatively short period of time, and ensures a constancy of flow of the fluid through the outlet by reducing any “dead time” where no fluid is being pumped. One skilled in the art would also appreciate that it is not necessary for the displacement devices to expel all of the fluid within the corresponding fluid chambers 62a, 62b. Instead, the amount of fluid to be displaced or pumped is equal to the volume displaced by the membrane by the deflection device.

While the embodiments described above are most economically feasible while permitting a completely disposable pump body 50, it may be necessary under specific circumstances to have a pump body 50 that is more conducive to continuity of flow. That is, the use of push points 76 may create a positive pressure, or bolus, that would act to displace a small volume of fluid in the forward direction upon activation to the extended position. Thus, to provide for a more stable flow while maintaining the disposable nature of the pump body 50, the push points 76 disposed within the fluid flow network 72 may be replaced with a valve, for example, a stop-cock style valve. As illustrated in FIGS. 6A-6D, the push points 76 of FIGS. 5A to 5D have been replaced with a stop-cock style valve 110 at each respective location. Generally, the stop-cock style valve 110 would be operated by a rotational-actuator 136 in a manner similar to the stepper motor 90 of FIG. 4. The stop-cock valve 110 is known to be a rotatable valve having a stem 114 and a flow passage 116. More particularly, the flow passage 116 extends substantially straight and transversely through the stem 114. Each stop-cock valve 110 includes a coupling portion corresponding to each stop-cock valve 110.

Rather than interrupting the fluid flow within the fluid flow network by applying pressure by a pressure device 102 at the raised portion within the fluid flow network 72, operation of the stop-cock valve 110 is effectuated by coupling to a rotational actuator 136 for rotating the stop-cock valve 110 between first and second positions, as subsequently discussed further. The rotational actuator 136 may be a stepper motor, as described previously with respect to the displacement device 92 and the pressure devices 102; however, other suitable rotational actuators 136 may be used within the scope of the present invention. For example, solenoid operated valves can be used in lieu of the stepper motors, or any other device can be used that is suitable for rotating the stop-cock valve 110 amongst the two positions.

One manner by which the rotational actuators 136 can be coupled to the stop-cock valves 110 is by a coupling portion. One example of a coupling portion, as illustrated in FIG. 8, includes an actuator tip 120 upon the actuator 136 that is received by a hollow portion 124 within the head 122 of the stop-cock 110. The hollow portion 124 can be formed as an Allen-wrench-style opening for receiving the Allen-wrench-style actuator tip 120. In this way, the head 122 of the stop-cock 110 is rotated by connection formed between the actuator tip 120 and the hollow portion 124. The head 122 may be formed as the same molding with the stem 114. Alternatively, the head 122 may be formed separately and coupled to the stem 124. The opposing end of the stem 124 may then be secured into the pump body 50, such as by a threaded end 126 and nut 128 as shown. Other means of securing the stop-cock valve 110 would be known and used as appropriate.

Valve 110 can be rotated by the corresponding rotational actuator 136 between a first position wherein the flow passage 116 of the valve 110 is in fluid communication with the fluid flow network 72 and a second position wherein the pump chamber 110 is not in fluid communication with the fluid flow network 72. Just as was described previously, in some detail with respect to push point interrupted flow of fluid, the actuators of stop-cock valves 110a-110f of FIGS. 6A-6D operate in a manner similar to FIGS. 5A-5D, respectively, the difference being that rather than activating a push point to the extended position, the stop-cock valve 110 is rotated from the first to the second position.

While the foregoing description has set forth various embodiments of the present invention in particular detail, it must be understood that numerous modifications, substitutions, and changes can be undertaken without departing from the true spirit and scope of the present invention as defined by the ensuing claims. For example, while the fluid flow network 72 of the illustrated embodiments includes six push points or stop-cock valves with a dual input and single output, the fluid flow networks according to the principles of the present invention can incorporate different numbers of valves and the valves can have different configurations, i.e., they may not be six push points. Also, while a plunger has been specifically described as the displacement device 92, any such mechanisms for displacing the member and associated fluid may be utilized. Pumps in accordance with the principles of the present invention can be used in a variety of applications, ranging from low to high volume fluid applications or low to high disposable costs. However, pumps in accordance with the principles of the present invention have particularly advantageous use in large volume fluid applications. The invention is therefore not limited to any specific embodiment as described, but is only limited as defined by the following claims.

What is claimed is:

1. A pump module for use in a medical fluid dispensing system, said pump module comprising:
a pump body having first and second portions;
first and second fluid chambers formed in at least one of said first and second portions of said pump body;
a fluid flow network formed in at least one of said first and second portions of said pump body for supplying fluid from a fluid source to said fluid chambers and for dispensing fluid from said chambers during operation of said pump module;
at least one membrane operably associated with said first and second fluid chambers; and
first and second actuators operably associated with said at least one membrane and with said first and second fluid chambers, respectively, for displacing said membrane and thereby displacing fluid from said first and second chambers, respectively.

2. The pump module of claim 1 wherein:
said first portion is a back portion and said second portion is a cover portion,
said first and second fluid chambers are formed in said back portion of said pump body,
said at least one membrane is disposed between said back portion and said cover portion,
said cover portion has first and second openings therein corresponding to said first and second fluid chambers, respectively, said first and second openings permitting said first and second actuators, respectively, to displace said membrane, and
said first and second actuators have first and second fluidically isolated displacement devices, respectively, which contact said membrane to displace fluid from said first and second fluid chambers, respectively.

3. The pump module of claim 2 wherein said fluidically isolated displacement devices are plungers.

4. The pump module of claim 2 wherein said first and second fluid chambers are recesses within said back portion.

5. The pump module of claim 1 wherein said pump body is constructed from a non-compliant material.

6. The pump module of claim 1 wherein said first and second actuators are independently operable from one another.

7. The pump module of claim 2 wherein said back portion of said pump body further includes at least one push point which, when pressed, interrupts fluid flow in said fluid flow network.

8. The pump module of claim 7 wherein:
the cover portion has an opening corresponding to said push point; and
a third displacement device operably associated with said membrane at said push point and said opening.

9. The pump module of claim 8 wherein said third displacement device has a fluidically isolated displacement device, which interrupts fluid flow in the fluid network.

10. The pump module of claim 2 wherein said cover portion of said pump body further includes at least one fluid valve which interrupts fluid flow in the fluid network.

11. The pump module of claim 2 wherein said first and second fluid chambers and said fluid flow network are fluidically sealed by said membrane.

12. The pump module of claim 11 wherein said membrane includes a first membrane to fluidically seal said first and second fluid chambers and a second membrane to fluidically seal said fluid flow network.

13. The pump module of claim 11 wherein said first and second fluid chambers are fluidically sealed with said membrane by an o-ring.

14. The pump module of claim 11 wherein an inner diameter of said first and second fluid chambers and an outer diameter of said displacement devices are constructed such that there is complete contact between said inner and outer diameters.

15. A pump module for use in a medical fluid dispensing system comprising:
a pump body having first and second portions;
first and second fluid chambers formed in at least one of said first and second portions of said pump body;
a fluid flow network formed in at least one of said first and second portions of said pump body for supplying fluid from a fluid source to said fluid chambers and for dispensing fluid from said chambers during operation of said pump module;
at least one membrane operably associated with said first and second fluid chambers;
first and second actuators operably associated with said at least one membrane and with said first and second fluid chambers, respectively, for displacing said membrane and thereby displacing fluid from said first and second chambers, respectively;
first and second openings formed in at least one of said first and second portions of said pump body, wherein said first and second openings spatially correspond with said first and second fluid chambers, respectively;
a first actuator operably associated with said membrane and said first chamber for displacing said membrane and thereby displacing fluid from said first chamber into said fluid flow network;
a second actuator operably associated with said membrane and said second chamber for displacing said membrane and thereby displacing fluid from said second chamber into said fluid flow network; and
at least one valve which, when activated, interrupts fluid flow in the fluid network.

16. A pump module as in claim 15, wherein said valve further includes a push point.

17. A pump module as in claim 15, wherein said valve further includes a stock cock valve system.

18. A method of manufacturing a pump module for use in a medical fluid dispensing system comprising:
using a non-compliant material to form a pump body having first and second portions;
forming first and second fluid chambers in at least one of said first and second portions of said pump body;
forming a fluid flow network in at least one of said first and second portions of said pump body for supplying fluid from a fluid source to said fluid chambers and for dispensing fluid from said chambers during operation of said pump module;
forming first and second openings in the other of the first and second portions of the pump body, wherein the first and second openings correspond to the first and second fluid chambers, respectively;
positioning at least one membrane between said first and second portions to fluidically seal said fluid chambers and said fluid flow network such that said membrane is operably associated with said fluid chambers and said fluid flow network;
providing first and second actuators positionally associated with said first and second fluid chambers, respectively, to be operably associated with said membrane for displacing said membrane and thereby displacing fluid from said first and second chambers;
positioning said membrane between said first and second portions of said pump body; and
securing said first and second portions of said pump body together.

19. A method as recited in claim 18, wherein the step of forming the fluid flow network comprises:
forming at least one push point in at least one of said first and second portions and within said fluid flow network.

20. A method as recited in claim 18, wherein the step of forming said first and second openings further includes constructing an inner diameter of said first and second openings and an outer diameter said first and second actuators such that substantially no clearance is formed between said inner and outer diameters.

21. A method as recited in claim 18, wherein the step of positioning the membrane further includes providing tension to said membrane such that said membrane is tightly held until said first and second portions are secured together.

22. A method for pumping fluid in a medical fluid dispensing system comprising the steps of:
providing a pump body having first and second portions where at least one of said first and second portions includes first and second fluid chambers and a fluid flow network and wherein at least one membrane is disposed between said first and second portions;
supplying a fluid through said fluid flow network to said fluid chambers;
displacing said membrane at said first and second fluid chambers thereby displacing fluid from said first and second chambers, through said fluid network, and out of said pump body.

23. A method as recited in claim 22, wherein the step of pumping further comprises:
initiating a first pumping cycle to displace at least a portion of the fluid out of said first fluid chamber; and
initiating a second pumping cycle, before the first pumping cycle is completed, to displace at least a portion of the fluid out of said second fluid chamber.

24. A method as recited in claim 23, wherein the step of pumping further comprises:
refilling said first fluid chamber after the completion of said first pumping cycle and during said second pumping cycle.

25. A method as recited in claim 24, wherein the step of pumping further comprises:
refilling said second pump chamber after the completion of said second pumping cycle and during a third pumping cycle.
